(19) United States
(12) Patent Application Publication
(10) Pub. No.: US 2007/0043217 A1
(43) Pub. Date: Feb. 22, 2007

(54) Method for the distillative separation of mixtures containing ethyleneamines

(75) Inventors: Markus Siegert, Heidelberg (DE); Johann-Peter Melder, Bohl-Iggelheim (DE); Thomas Krug, Worms (DE); Jan Nouwen, Brecht (BE)

Correspondence Address: CONNOLLY BOVE LODGE & HUTZ, LLP, P O BOX 2207, WILMINGTON, DE, 19899, US

(73) Assignee: BASF Aklengesellschaft, Ludwigshafen (DE)

(21) Appl. No.: 10/574,680

(22) PCT Filed: Oct. 15, 2004
(86) PCT No.: PCT/EP04/11584

§ 371 Date: Apr. 4, 2006

(30) Foreign Application Priority Data
Oct. 17, 2003 (DE) 10349059.0

(51) Int. Cl. C07D 241/04; C07C 213/02

(52) U.S. Cl. 544/358; 564/503; 564/511

(57) ABSTRACT

A process for distillatively separating mixtures comprising ethylenamines, wherein the separation is carried out in one or more dividing wall columns and wherein the ethylenamines are in particular ethylenediamine (EDA), piperazine (PIP), diethylenetriamine (DETA), aminoethylethanolamine (AEEA) and/or monoethanolamine (MEOA).

The present invention relates to a process for distillatively separating mixtures comprising ethylenamines.

For the distillative, for example continuous, separation of multisubstance mixtures, various process variants can be used. In the simplest case, the mixture to be separated (feed mixture) is separated into two fractions, a low-boiling top fraction and a high-boiling bottom fraction.

When feed mixtures are separated into more than two fractions, a plurality of distillation columns has to be used in this process variant. In order to restrict the apparatus demands, columns having liquid or vaporous side draws are used if possible in the separation of multisubstance mixtures.

However, the opportunity to employ distillation columns having side draws is highly restricted by the fact that products withdrawn at the side draw points are rarely if ever completely pure. In the case of side withdrawals in the rectifying section of the column, which are typically in liquid form, the side product still contains fractions of low-boilers which should be removed via the top. The same applies to side withdrawals in the stripping section of the column, which are usually in vaporous form, in which the side product still has high boiler fractions.

The use of conventional side draw columns is therefore restricted to cases in which contaminated side products are permissible.

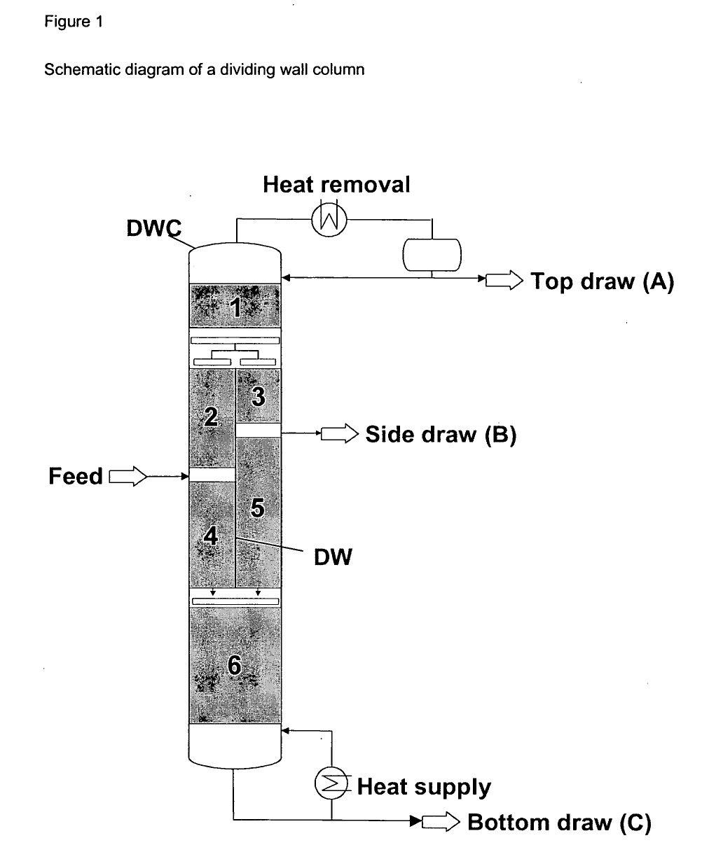

One means of remedy is offered by dividing wall columns (see, for example, FIG. 1). This column type is described, for example, in:

U.S. Pat. No. 2,471,134, U.S. Pat. No. 4,230,533, EP-A-122 367, EP-A-126 288, EP-A-133 510,

Chem. Eng. Technol. 10, (1987), pages 92-98,

Chem.-Ing.-Tech. 61, (1989), No. 1, pages 16-25,

Gas Separation and Purification 4 (1990), pages 109-114,

Process Engineering 2 (1993), pages 33-34,

Trans IChemE 72 (1994), Part A, pages 639-644, and

Chemical Engineering 7 (1997), 72-76.

In this design, it is possible to withdraw side products likewise in pure form. Disposed in the middle region above and below the feed point and the side withdrawal is a dividing wall which seals the feed section from the withdrawal section and prevents transverse mixing of liquid and vapor streams in this column section. This reduces the total number of distillation columns required in the separation of multisubstance mixtures. Since this column type constitutes a simplification in apparatus terms of thermally coupled distillation columns, it additionally has particularly low energy consumption. A description of thermally coupled distillation columns which may be designed in different apparatus configuration can likewise be found in the abovementioned references in the technical literature.

Dividing wall columns and thermally coupled distillation columns offer advantages compared to the arrangement of conventional distillation columns both with regard to the energy demands and the capital costs, and are therefore being used to an increasing extent in industry.

For the control of dividing wall columns and thermally coupled columns, various control strategies are described. Descriptions can be found in:

U.S. Pat. No. 4,230,533, DE-C2-35 22 234, EP-A-780 147,

Process Engineering 2 (1993), 33-34, and

Ind. Eng. Chem. Res. 34 (1995), 2094-2103.

The prior German patent application No. 10335991.5 of Aug. 1, 2003 relates to a process for preparing ethylenamines by reacting monoethanolamine (MEOA) with ammonia in the presence of a catalyst and separating the resulting reaction effluent in distillation columns.

It is an object of the present invention to provide an improved economically viable process for separating mixtures comprising ethylenamines. The individual ethylenamines, especially ethylenediamine (EDA), piperazine (PIP), diethylenetriamine (DETA) and aminoethylethanolamine (AEEA) should be obtained in high purity and quality (for example color quality).

We have found that this object is achieved by a process for distillatively separating mixtures comprising ethylenamines, which comprises carrying out the separation in one or more dividing wall columns.

The ethylenamines to be separated are in particular EDA, PIP, DETA, AEEA and/or monoethanolamine (MEOA).

The mixture comprising ethylenamines is preferably a product which is obtained by reacting MEOA with ammonia and subsequently partly or fully removing ammonia and water.

For example, EDA, DETA, PIP and AEEA may be prepared from MEOA and ammonia by the processes described in U.S. Pat. No. 2,861,995 (Dow), DE-A-1 172 268 (BASF) and U.S. Pat. No. 3,112,318 (Union Carbide), (cf. Ullmann's Encyclopedia of Industrial Chemistry, 6th Edition, 2000 Electronic Release, Chapter 8.1.1: 1,2-Diaminoethane), in which ammonia is used, for example, in a from one- to twenty-fold molar excess and, for example, from 40 to 60% of the MEOA is converted. The effluent mixture of these reactions, consisting predominantly of ammonia, water, MEOA, EDA, DETA, PIP, AEEA and higher-boiling ethylenamines and ethylenamino alcohols, is initially decompressed and degassed, and ammonia and water are subsequently removed by distillation. The process according to the invention is especially suitable for the further continuous workup of the mixture of EDA, PIP, (unconverted) MEOA, DETA, AEEA and further higher-boiling byproducts which remains after the dewatering.

A typical dividing wall column (DWC) to be employed in the process according to the invention (see FIG. 1) in each case has a dividing wall (DW) in the longitudinal direction of the column to form an upper combined column region (1), a lower combined column region (6), a feed section (2, 4) having rectifying section (2) and stripping section (4), and also a withdrawal section (3, 5) having rectifying section (3) and stripping section (5), and the mixture to be separated (feed) is fed in the middle region of the feed section (2, 4), the high boiler fraction is removed via the bottom (bottom draw C), the low boiler fraction is removed via the top (top draw A) and the medium boiler fraction is removed from the middle region of the withdrawal section (3, 5) (side draw B).

The dividing wall column(s) of the process according to the invention has/each have preferably from 30 to 100, in particular from 50 to 90, theoretical plates.

The mixture comprising ethylenamines is preferably worked up in a dividing wall column in which EDA, especially EDA having a purity of >99.0% by weight, is obtained as a top product, and PIP, especially PIP having a purity of >99.0% by weight, is obtained as a side draw stream at an operating pressure of generally from 0.1 to 5 bar, preferably from 0.3 to 2 bar, more preferably from 0.7 to 1.6 bar.

In this document, “operating pressure” refers to the absolute pressure measured at the top of the column.

After the removal of EDA and PIP, preference is given to effecting further workup in a dividing wall column in which MEOA is obtained as a top product and DETA, especially DETA having a purity of >99.0% by weight, is obtained as a side draw stream at an operating pressure of generally from 0.01 to 2.5 bar, preferably from 0.01 to 0.70 bar, in particular from 0.05 to 0.25 bar.

After the removal of EDA, PIP, MEOA and DETA, preference is given to effecting further workup in a dividing wall column in which AEEA, especially AEEA having a purity of >99.0% by weight, is obtained as a side draw stream at an operating pressure of generally from 0.001 to 1.0 bar, preferably from 0.001 to 0.05 bar, in particular from 0.005 to 0.025 bar.

The dividing wall columns are in particular connected in such a way that the crude mixture from the synthesis of ethylenamines, after the partial or complete removal of ammonia and water, is fed to the first dividing wall column in which pure EDA is obtained as a top product and pure PIP as a side draw stream, and that the bottom stream of this column is worked up further in the second dividing wall column in which MEOA is obtained as a top product and pure DETA as a side draw stream, and the bottom stream of the second dividing wall column is fed to a third dividing wall column in which pure AEEA is obtained as a side draw stream.

The bottom product of the dividing wall column for obtaining AEEA is preferably worked up further in one or more further conventional distillation columns to concentrate and purify further higher-boiling ethylenamines and/or ethylenamino alcohols.

Higher-boiling ethylenamines and/or ethylenamino alcohols here are those amines which (at the same pressure) have a higher boiling point than AEEA.

In an alternative procedure, the bottom stream of the above-detailed dividing wall column for removing EDA and PIP is worked up further in further conventional distillation columns to obtain first MEOA as a top product in one distillation column and, from the bottom stream of this column, DETA, especially DETA having a purity of >99.0% by weight, is obtained as a top product, and the bottom stream of this column is
(a) fed to one or more further conventional columns in order to obtain AEEA, especially AEEA having a purity of >99.0% by weight, or
(b) fed to a dividing wall column in which AEEA, especially AEEA having a purity of >99.0% by weight, is obtained as a side draw stream.

In a further alternative procedure, the mixture comprising ethylenamines is fed to a conventional distillation column in which an EDA/PIP mixture is obtained as a top product, and is separated in a further conventional column into EDA, especially EDA having a purity of >99.0% by weight, and PIP, especially PIP having a purity of >99.0% by weight, and the bottom stream of this column is worked up further in a dividing wall column in such a way that MEOA is obtained as a top product and DETA, especially DETA having a purity of >99.0% by weight, is obtained as a side draw stream, and the bottom stream of this dividing wall column is
(a) fed to one or more conventional distillation columns in order to obtain AEEA, especially AEEA having a purity of >99.0% by weight, or
(b) fed to a further dividing wall column in which AEEA, especially AEEA having a purity of >99.0% by weight, is obtained as a side draw stream.

In particular, the upper combined column region (1) of the dividing wall column (DWC) for removing EDA and PIP in the process according to the invention has from 5 to 50%, preferably from 20 to 35%, the rectifying section (2) of the feed section (2, 4) of the column has from 5 to 50%, preferably from 10 to 20%, the stripping section (4) of the feed section of the column has from 5 to 50%, preferably from 20 to 35%, the rectifying section (3) of the withdrawal section (3, 5) of the column has from 5 to 50%, preferably from 7 to 20%, the stripping section (5) of the withdrawal section of the column has from 5 to 50%, preferably from 20 to 35%, and the combined lower region (6) of the column has from 5 to 50%, preferably from 20 to 35%, of the total number of theoretical plates of the column.

In particular, the upper combined column region (1) of the dividing wall column (DWC) for removing MEOA and DETA in the process according to the invention has from 5 to 50%, preferably from 5 to 15%, the rectifying section (2) of the feed section (2, 4) of the column has from 5 to 50%, preferably from 25 to 40%, the stripping section (4) of the feed section of the column has from 5 to 50%, preferably from 20 to 35%, the rectifying section (3) of the withdrawal section (3, 5) of the column has from 5 to 50%, preferably from 15 to 25%, the stripping section (5) of the withdrawal section of the column has from 5 to 50%, preferably from 40 to 55%, and the combined lower region (6) of the column has from 5 to 50%, preferably from 15 to 25%, of the total number of theoretical plates of the column.

In particular, the upper combined column region (1) of the dividing wall column (DWC) for removing AEEA in the process according to the invention has from 5 to 50%, preferably from 5 to 30%, the rectifying section (2) of the feed section (2, 4) of the column has from 5 to 50%, preferably from 15 to 35%, the stripping section (4) of the feed section of the column has from 5 to 50%, preferably from 15 to 35%, the rectifying section (3) of the withdrawal section (3, 5) of the column has from 5 to 50%, preferably from 15 to 35%, the stripping section (5) of the withdrawal section of the column has from 5 to 50%, preferably from 15 to 35%, and the combined lower region (6) of the column has from 5 to 50%, preferably from 10 to 25%, of the total number of theoretical plates of the column.

In particular, the sum of the number of theoretical plates of the subregions (2) and (4) in the feed section in the dividing wall column (DWC) is from 80 to 110%, preferably from 90 to 100%, of the sum of the number of plates of the subregions (3) and (5) in the withdrawal section.

In the process according to the invention, the feed point and the side draw point of the dividing wall column for removing EDA and PIP are preferably disposed at a different height in the column with regard to the position of the theoretical plates by the feed point differing from the side draw point by from 1 to 10, in particular from 1 to 5, theoretical plates.

In the process according to the invention, the feed point and the side draw point of the dividing wall column for removing MEOA and DETA are preferably disposed at a different height in the column with regard to the position of the theoretical plates by the feed point differing from the side draw point by from 1 to 20, in particular from 5 to 15, theoretical plates.

In the process according to the invention, the feed point and the side draw point of the dividing wall column for removing AEEA are preferably disposed at a different height in the column with regard to the position of the theoretical plates by the feed point differing from the side draw point by from 1 to 20, in particular from 5 to 15, theoretical plates.

If particularly high requirements are placed on the purities of the products, it is favorable to provide the dividing wall with thermal insulation. A description of the different means of thermally insulating the dividing wall can be found in EP-A-640 367. A jacketed design with an interstitial narrow gas space is particularly favorable.

The subregion of the column (DWC) which is divided by the dividing wall (DW) and consists of the subregions 2, 3, 4 and 5 or parts thereof is preferably charged with structured packings or random packings and the dividing wall is designed with heat insulation in these subregions.

Alternatively, the subregion of the column (DWC) which is divided by the dividing wall (DW) and consists of the subregions 2, 3, 4 and 5 or parts thereof is preferably charged with trays and the dividing wall is designed with heat insulation in these subregions.

In the process according to the invention, the medium boiler fraction is withdrawn in liquid form or gaseous form at the side draw point.

The vapor flow rate at the lower end of the dividing wall (DW) is preferably adjusted by the selection and/or dimensioning of the separating internals and/or the installation of pressure drop-inducing apparatus, for example of perforated plates, in such a way that the ratio of the vapor flow rate in the feed section to that of the withdrawal section is from 0.8 to 1.2, in particular from 0.9 to 1.1

The ratios mentioned in this document which relate to certain streams (for example liquid streams, vapor streams, bottom streams, feed streams, side draw streams) are based on the weight.

The liquid descending out of the upper combined region (1) of the column is preferably collected in a collecting chamber disposed in the column or outside the column and is precisely divided by a fixed setting or control at the upper end of the dividing wall (DW) in such a way that the ratio of the liquid flow rate to the feed section to that to the stripping section is from 0.1 to 1.0, in particular from 0.25 to 0.8.

In the process according to the invention, the liquid is preferably conveyed to the feed section (feed) via a pump or is introduced with flow control using a static feed head of at least 1 m, and the control is adjusted in such a way that the amount of liquid introduced to the feed section cannot fall below 30% of the normal value.

In the process according to the invention, the division of the liquid descending out of the subregion 3 in the withdrawal section of the column to the side draw and to the subregion 5 is preferably adjusted by a control in the withdrawal section of the column in such a way that the amount of liquid introduced to the subregion 5 cannot fall below 30% of the normal value

It is also preferred that the dividing wall column (DWC) has sampling means at the upper and lower end of the dividing wall (DW) and liquid or gaseous samples are taken from the column continuously or at time intervals and investigated with regard to their composition.

In the process according to the invention, the division ratio of the liquid at the upper end of the dividing wall (DW) is preferably adjusted in such a way that the concentration of those components of the high boiler fraction for which a certain limiting value for the concentration is to be achieved in the side draw, in the liquid at the upper end of the dividing wall, is from 5 to 75%, in particular from 5 to 40%, of the value which is to be achieved in the side draw product, and the liquid division is adjusted to the effect that more liquid is passed to the feed section at higher contents of components of the high boiler fraction, and less liquid at lower contents of components of the high boiler fraction.

In the process according to the invention, the heating output in the evaporator is preferably adjusted in such a way that the concentration of those components of the low boiler fraction for which a certain limiting value for the concentration is to be achieved in the side draw, at the lower end of the dividing wall (DW), is adjusted in such a way that the concentration of components of the low boiler fraction in the liquid at the lower end of the dividing wall is from 10 to 99%, preferably from 25 to 97.5%, of the value which is to be achieved in the side draw product, and the heating output is adjusted to the effect that the heating output is increased at a higher content of components of the low boiler fraction and the heating output is reduced at a lower content of components of the low boiler fraction.

In the process according to the invention, the distillate is preferably withdrawn under temperature control and the control temperature used is a measurement point in the subregion 1 of the column which is disposed from 2 to 20, in particular from 4 to 15, theoretical plates below the upper end of the column.

In the process according to the invention, the bottom product is preferably withdrawn under temperature control and the control temperature used is a measurement point in the subregion 6 of the column which is disposed from 2 to 20, in particular from 4 to 15, theoretical plates above the lower end of the column.

In a further particular embodiment, the side product in the side draw is withdrawn under level control and the control part used is the liquid level in the evaporator.

In a further inventive variation of the process for distillatively working up ethylenamines, instead of one of the dividing wall columns mentioned, a connection of two distillation columns in the form of a thermal coupling is used.

The two thermally coupled distillation columns are each preferably equipped with a dedicated evaporator and condenser.

Moreover, the two thermally coupled columns are preferably operated at different pressures and only liquids are conveyed in the connection streams between the two columns.

In the case of the connection of two distillation columns, the bottom stream of the first column is preferably partly or fully evaporated in an additional evaporator and subsequently fed to the second column in biphasic form or in the form of a gaseous and of a liquid stream.

In particular, the feed stream (feed) to the column (DWC or distillative column without DW) is partly or fully preevaporated and is fed to the column in biphasic form or in the form of a gaseous and of a liquid stream.

The dividing wall is preferably not welded into the column, but rather is configured in the form of loosely inserted and adequately sealed subsegments.

The aforementioned loose dividing wall preferably has internal manholes or removable segments which allow access from one side of the dividing wall to the other side within the column.

The liquid distribution in the individual subregions of the column (DWC) may preferably be deliberately adjusted in a nonuniform manner.

The liquid is preferably introduced to an increased extent in the wall region in the subregions 2 and 5 and the liquid is preferably introduced to a reduced extent in the wall region in the subregions 3 and 4.

As already mentioned, dividing wall columns may also be replaced in the process according to the invention by in each case two thermally coupled columns. This is favorable in particular when the columns are already available or the columns are to be operated at different pressures. In the case of thermally coupled columns, it may be advantageous to partly or fully evaporate the bottom stream of the first column in an additional evaporator and then to feed it to the second column. This preevaporation is an option especially when the bottom stream of the first column contains relatively large amounts of medium boilers. In this case, the preevaporation may be effected at a lower temperature level and the evaporator of the second column deburdened. Moreover, this measure substantially deburdens the stripping section of the second column. The preevaporated stream may be fed to the second column in biphasic form or in the form of two separate streams.

In addition, both in the case of dividing wall columns and in the case of thermally coupled columns, it may be advantageous to subject the feed stream to a preevaporation and subsequently feed it to the column in biphasic form or in the form of two streams. This preevaporation is an option particularly when the feed stream contains relatively large amounts of low boilers. The preevaporation may substantially deburden the stripping section of the column.

Dividing wall columns and thermally coupled columns may either be designed as packed columns having random packings or structured packings or as tray columns.

In the purifying distillation of DETA and recovery of MEOA mentioned, which are preferably operated under reduced pressure, it is recommended to use packed columns. Structured sheet metal packings having a specific surface area of from 100 to 500 m²/m³, preferably from about 250 to 350 m²/m³, are particularly suitable.

In the purifying distillation of EDA and PIP, which are preferably operated at pressures slightly above atmospheric pressure so that the temperature in all regions of the column is slightly above the melting temperature of PIP, either trays or packings may be used. Suitable trays are in particular valve trays. In the case of packings, structured sheet metal packings having a specific surface area of from 100 to 500 m²/m³, preferably from about 250 to 350 m²/m³, are particularly suitable.

The purifying distillation of AEEA is preferably carried out under reduced pressure, and it is therefore recommended here also to use packings as separating internals. Structured sheet metal packings having a specific surface area of from 100 to 500 m² μm³, preferably from about 250 to 350 m²/m³, are particularly suitable.

In the case of the separation of multisubstance mixtures into a low boiler, medium boiler and high boiler fraction, there typically exist specifications of the maximum permissible fraction of low boilers and high boilers in the medium boiler fraction. In this context, either individual components which are critical to the separating problem, known as key components, or the sum of a plurality of key components, is specified.

The compliance with the specification for the high boilers in the medium boiler fraction is controlled via the division ratio of the liquid at the upper end of the dividing wall. The division ratio of the liquid at the upper end of the dividing wall is adjusted in such a way that the concentration of the key components for the high boiler fraction in the liquid at the upper end of the dividing wall is from 10 to 80%, preferably from 30 to 50%, of the value which is to be attained in the side draw product, and the liquid division is adjusted to the effect that more liquid is passed to the feed section at higher contents of key components in the high boiler fraction and less liquid is passed to the feed section at lower contents of key components in the high boiler fraction.

Accordingly, the specification for the low boilers in the medium boiler fraction is controlled via the heating output. In this case, the heating output in the evaporator is adjusted in such a way that the concentration of key components of the low boiler fraction in the liquid at the lower end of the dividing wall is from 10 to 80%, preferably from 30 to 50%, of the value which is to be attained in the side draw product, and the heating output is adjusted to the effect that the heating output is increased at a higher content of key components in the low boiler fraction and the heating output is reduced at a lower content of key components in the low boiler fractions.

To compensate for disruptions in the feed rate or in the feed concentration, it is additionally found to be advantageous to ensure, by appropriate control methods in the process control system, that the flow rates of the liquids which are introduced to the column parts 2 and 5 (cf. FIG. 1) can never fall to below 30% of their normal value.

Suitable for withdrawing and dividing the liquids at the upper end of the dividing wall and at the side withdrawal point are collecting chambers, either internal or disposed outside the column, for the liquid which assume the function of a pump reservoir or ensure sufficiently high static liquid head, which enable liquid to be passed on in a controlled manner by control elements, for example valves. When packed columns are used, the liquid is initially collected in collectors and passed from there into an internal or external collecting chamber.

Instead of a dividing wall column, which is preferable in the case of new construction with regard to the capital costs, it is also possible to connect two distillation columns by a type of thermal coupling in such a way that they correspond to a dividing wall column with regard to the energy demands.

When existing columns are available, they may be a sensible alternative to dividing wall columns. The most suitable forms of the connection may be selected depending on the number of theoretical plates of the available columns. It is possible to select connection forms which allow only liquid connecting streams to occur between the individual distillation columns. These specific connections offer the advantage that the two distillation columns may be operated under different pressures with the advantage that they can be better adapted to the temperature levels of heating and cooling energies present. In general, the pressure selected in the column at which the low boiler fraction is withdrawn is from about 0.5 to 1.0 bar higher than in the column at which the high boiler fraction is withdrawn.

### EXAMPLE

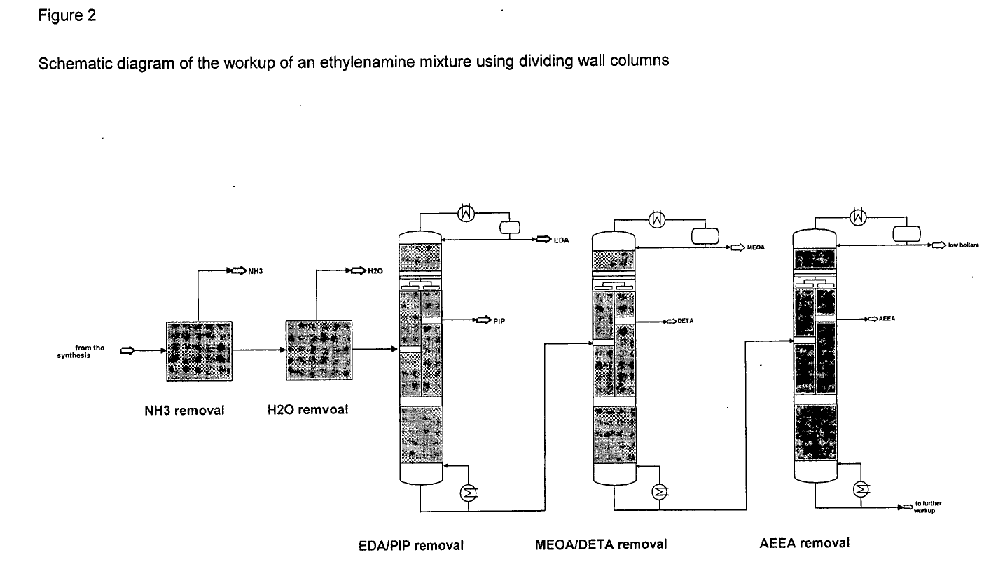

FIG. 2 shows, as an example, the separation of an ethylenamine synthesis mixture, after preceding removal of ammonia and water, into pure ethylenediamine product (EDA), pure piperazine product (PIP) and a high boiler fraction. The high boiler fraction is separated in a further distillation column into monoethanolamine (MEOA), pure diethylenetriamine product (DETA) and a high boiler fraction. Last but not least, pure aminoethylethanolamine product (AEEA) and a further high boiler fraction are obtained in a third dividing wall column from the high boiler fraction which is obtained at the bottom of the second dividing wall column. Any low boilers which are present and are undesired in the AEEA are removed via the top of the column.

1. A process for distillatively separating mixtures comprising ethylenamines, wherein the mixture comprising ethyleneamine is a product obtained by reacting monoethanolamine (MEOA) with ammonia and subsequently partly or fully removing ammonia and water, the ethyleneamines are ethylenediamine (EDA), piperazine (PIP), diethylenetriamine (DETA), aminoethylethanolarnine (AEEA) and/or monoethanolamine (MEOA) and the separation is carried out in one or more dividing wall columns.

2. The process according to claim 1, wherein the dividing wall column (DWC) in each case has a dividing wall (DW) in the longitudinal direction of the column to form an upper combined column region (1), a lower combined column region (6), a feed section (2, 4) having rectifying section (2) and stripping section (4), and also a withdrawal section (3, 5) having rectifying section (3) and stripping section (5), and the mixture to be separated (feed) is fed in the middle region of the feed section (2, 4), the high boiler fraction is removed via the bottom (bottom draw C), the low boiler fraction is removed via the top (top draw A) and the medium boiler fraction is removed from the middle region of the withdrawal section (3, 5) (side draw B).

3. The process according to claim 1, wherein the dividing wall column has from 30 to 100 theoretical plates or the dividing wall columns each have from 30 to 100 theoretical plates.

4. The process according to claim 1, wherein the mixture comprising ethylenamines is worked up in a dividing wall column in which EDA is obtained as a top product and PIP as a side draw stream at an operating pressure, which is understood to mean the absolute pressure measured at the top of the column, of from 0.1 to 5 bar.

5. The process according to claim 1, wherein, after the removal of EDA and PIP, further workup is effected in a dividing wall column in which MEOA is obtained as a top product and DETA as a side draw stream at an operating pressure, which is understood to mean the absolute pressure measured at the top of the column, of from 0.01 to 2.5 bar.

6. The process according to claim 1, wherein, after the removal of EDA, PIP, MEOA and DETA, further workup is effected in a dividing wall column in which AEEA is obtained as a side draw stream at an operating pressure, which is understood to mean the absolute pressure measured at the top of the column, of from 0.001 to 1.0 bar.

7. The process according to claim 6, wherein the bottom product of the dividing wall column for obtaining AEEA is worked up further in one or more conventional distillation columns to concentrate and purify further higher-boiling ethylenamines and/or ethylenamino alcohols.

8. The process according to claim 4, wherein the bottom stream of the dividing wall column is worked up further in further conventional distillation columns to obtain first MEOA as a top product in one distillation column and, from the bottom stream of this column, in the next column, DETA is obtained as a top product, and the bottom stream of this column is fed to one or more further conventional columns in order to obtain AEEA, or the bottom stream of this column is fed to a dividing wall column in which AEEA is obtained as a side draw stream.

9. The process according to claim 1, wherein the mixture comprising ethylenamines is fed to a conventional distillation column in which an EDA/PIP mixture is obtained as a top product, and is separated in a further conventional column into EDA and PIP, and the bottom stream of this column is worked up further in a dividing wall column in such a way that MEOA is obtained as a top product and DETA is obtained as a side draw stream, and the bottom stream of this dividing wall column is fed to one or more conventional distillation columns in order to obtain AEEA, or the bottom stream of this dividing wall column is fed to a further dividing wall column in which AEEA is obtained as a side draw stream.

10. The process according to claim 4, wherein the upper combined column region (1) of the dividing wall column (DWC) for removing EDA and PIP has from 5 to 50%, the rectifying section (2) of the feed section (2, 4) of the column has from 5 to 50%, the stripping section (4) of the feed section of the column has from 5 to 50%, the rectifying section (3) of the withdrawal section (3, 5) of the column has from 5 to 50%, the stripping section (5) of the withdrawal section of the column has from 5 to 50% and the combined lower region (6) of the column has from 5 to 50%, of the total number of theoretical plates of the column.

11. The process according to claim 5, wherein the upper combined column region (1) of the dividing wall column (DWC) for removing MEOA and DETA has from 5 to 50%, the rectifying section (2) of the feed section (2, 4) of the column has from 5 to 50%, the stripping section (4) of the feed section of the column has from 5 to 50%, the rectifying section (3) of the withdrawal section (3, 5) of the column has from 5 to 50%, the stripping section (5) of the withdrawal section of the column has from 5 to 50% and the combined lower region (6) of the column has from 5 to 50%, of the total number of theoretical plates of the column.

12. The process according to claim 6, wherein the upper combined column region (1) of the dividing wall column (DWC) for removing AEEA has from 5 to 50%, the rectifying section (2) of the feed section (2, 4) of the column has from 5 to 50%, the stripping section (4) of the feed section of the column has from 5 to 50%, the rectifying section (3) of the withdrawal section (3, 5) of the column has from 5 to 50%, the stripping section (5) of the withdrawal section of the column has from 5 to 50% and the combined lower region (6) of the column has from 5 to 50%, of the total number of theoretical plates of the column.

13. The process according to claim 1, wherein the sum of the number of theoretical plates of the subregions (2) and (4) in the feed section in the dividing wall column (DWC) is from 80 to 110% of the sum of the number of plates of the subregions (3) and (5) in the withdrawal section.

14. The process according to claim 4, wherein the feed point and the side draw point of the dividing wall column for removing EDA and PIP are disposed at a different height in the column with regard to the position of the theoretical plates by the feed point differing from the side draw point by from 1 to 10 theoretical plates.

15. The process according to claim 5, wherein the feed point and the side draw point of the dividing wall column for removing MEOA and DETA are disposed at a different height in the column with regard to the position of the theoretical plates by the feed point differing from the side draw point by from 1 to 20 theoretical plates.

16. The process according to claim 6, wherein the feed point and the side draw point of the dividing wall column for removing AEEA are disposed at a different height in the column with regard to the position of the theoretical plates by the feed point differing from the side draw point by from 1 to 20 theoretical plates.

17. The process according to claim 2, wherein the subregion of the column (DWC) which is divided by the dividing wall (DW) and consists of the subregions 2, 3, 4 and 5 or parts thereof is charged with structured packings or random packings and the dividing wall is designed with heat insulation in these subregions.

18. The process according to claim 2, wherein the subregion of the column (DWC) which is divided by the dividing wall (DW) and consists of the subregions 2, 3, 4 and 5 or parts thereof is charged with trays and the dividing wall is designed with heat insulation in these subregions.

19. A process according to claim 2, wherein the medium boiler fraction is withdrawn in liquid form at the side draw point.

20. The process according to claim 2, wherein the medium boiler fraction is withdrawn in gaseous form at the side draw point.

21. The process according to claim 2, wherein the vapor flow rate at the lower end of the dividing wall (DW) is adjusted by the selection and/or dimensioning of the separating internals and/or the installation of pressure drop-inducing apparatus in such a way that the ratio of the vapor flow rate in the feed section to that of the withdrawal section is from 0.8 to 1.2.

22. The process according to claim 2, wherein the liquid descending out of the upper combined region (1) of the column is collected in a collecting chamber disposed in the column or outside the column and is precisely divided by a fixed setting or control at the upper end of the dividing wall (DW) in such a way that the ratio of the liquid flow rate to the feed section to that to the stripping section is from 0.1 to 1.0.

23. The process according to claim 2, wherein the liquid is conveyed to the feed section (feed) via a pump or is introduced with flow control using a static feed head of at least 1 m, and the control is adjusted in such a way that the amount of liquid introduced to the feed section cannot fall below 30% of the normal value.

24. The process according to claim 2, wherein the division of the liquid descending out of the subregion 3 in the withdrawal section of the column to the side draw and to the subregion 5 is adjusted by a control in the withdrawal section of the column in such a way that the amount of liquid introduced to the subregion 5 cannot fall below 30% of the normal value.

25. The process according to claim 1, wherein the dividing wall column (DWC) has sampling means at the upper and lower end of the dividing wall (DW) and liquid or gaseous samples are taken from the column continuously or at time intervals and investigated with regard to their composition.

26. The process according to claim 1, wherein the division ratio of the liquid at the upper end of the dividing wall (DW) is adjusted in such a way that the concentration of those components of the high boiler fraction for which a certain limiting value for the concentration is to be achieved in the side draw, in the liquid at the upper end of the dividing wall, is from 5 to 75% of the value which is to be achieved in the side draw product, and the liquid division is adjusted to the effect that more liquid is passed to the feed section at higher contents of components of the high boiler fraction, and less liquid at lower contents of components of the high boiler fraction.

27. The process according to claim 2, wherein the heating output in the evaporator is adjusted in such a way that the concentration of those components of the low boiler fraction for which a certain limiting value for the concentration is to be achieved in the side draw, at the lower end of the dividing wall (DW), is adjusted in such a way that the concentration of components of the low boiler fraction in the liquid at the lower end of the dividing wall is from 10 to 99% of the value which is to be achieved in the side draw product, and the heating output is adjusted to the effect that the heating output is increased at a higher content of components of the low boiler fraction and the heating output is reduced at a lower content of components of the low boiler fraction.

28. The process according to claim 2, wherein the distillate is withdrawn under temperature control and the control temperature used is a measurement point in the subregion 1 of the column which is disposed from 2 to 20 theoretical plates below the upper end of the column.

29. The process according to claim 2, wherein the bottom product is withdrawn under temperature control and the control temperature used is a measurement point in the subregion 6 of the column which is disposed from 2 to 20 theoretical plates above the lower end of the column.

30. The process according to claim 2, wherein the side product in the side draw is withdrawn under level control and the control part used is the liquid level in the evaporator.

31. The process according to claim 1, wherein, instead of a dividing wall column, a connection of two distillation columns in the form of a thermal coupling is used.

32. The process according to claim 31, wherein the two thermally coupled distillation columns are each equipped with a dedicated evaporator and condenser.

33. The process according to claim 31, wherein the two thermally coupled columns are operated at different pressures and only liquids are conveyed in the connection streams between the two columns.

34. The process according to claim 31, wherein the bottom stream of the first column is partly or fully evaporated in an additional evaporator and subsequently fed to the second column in biphasic form or in the form of a gaseous and of a liquid stream.

35. The process according to claim 1, wherein the feed stream to the column (feed) is partly or fully preevaporated and is fed to the column in biphasic form or in the form of a gaseous and of a liquid stream.

36. A process according to claim 1, wherein the dividing wall is not welded into the column, but rather is configured in the form of loosely inserted and adequately sealed subsegments.

37. The process according to claim 36, wherein the loose dividing wall has internal manholes or removable segments which allow access from one side of the dividing wall to the other side within the column.

38. The process according to claim 2, wherein the liquid distribution in the individual subregions of the column (DWC) may be deliberately adjusted in a nonuniform manner.

39. The process according to claim 38, wherein the liquid is introduced to an increased extent in the wall region in the subregions 2 and 5 and the liquid is introduced to a reduced extent in the wall region in the subregions 3 and 4.

40-41. (canceled)
